(19) United States
(12) Patent Application Publication
(10) Pub. No.: US 2020/0016292 A1
(43) Pub. Date: Jan. 16, 2020

(54) DEVICE FOR DELIVERY OF POWDERED HEMOSTATIC AGENTS

(71) Applicants: BAXTER INTERNATIONAL INC., Deerfield, IL (US); BAXTER HEALTHCARE SA, Glattpark (Opfikon) (CH)

(72) Inventors: Russel L. HOLSCHER, Winnetka, IL (US); Paul Jeffrey SANDERS, Greendale, WI (US); Timothy Michael FULGHUM, Lakemoor, IL (US); John Joseph Anthony BARRY, Northbrook, IL (US)

(21) Appl. No.: 16/491,406

(22) PCT Filed: Mar. 7, 2018
(86) PCT No.: PCT/US2018/021313

§ 371 Date: Sep. 5, 2019

Related U.S. Application Data
(60) Provisional application No. 62/468,797, filed on Mar. 8, 2017.

(51) Int. Cl. A61L 26/00; A61K 38/17; A61K 38/48; A61K 38/36

(52) U.S. Cl. CPC A61L 26/0052; A61L 26/009; A61L 26/0066; A61K 38/36; A61K 38/4833; A61K 38/363; A61K 38/1703

(57) ABSTRACT

The disclosure provides a multilayered hemostatic device including a first layer including carboxymethyl cellulose, a second layer including a powdered hemostatic agent provided on the first layer, and a third layer including at least one of gauze, oxidized cellulose, or collagen provided on the second layer, wherein the first layer and the third layer encapsulate the second layer and the hemostatic agent comprises one of fibrinogen, thrombin, fibrin monomers, or the combination of thrombin and gelatin.

### FIELD OF THE INVENTION

The disclosure relates generally to multilayered hemostatic devices. More particularly, the disclosure relates to multilayered hemostatic devices including a powdered hemostat and a rapidly dissolving/degrading film.

### BACKGROUND

Hemostatic agents and sealants are currently used as an aid to stop bleeding, including hemorrhaging during surgery. Hemostatic agents are typically presented in the form of solid fabrics, powders or granules, or as liquids. Flowable hemostatic agents provide for good contact with irregular surfaces which are typical of wounds so that good hemostasis can be achieved. However, the flowable nature of particulate hemostatic agents also renders them relatively difficult to handle in use. Current powdered hemostats are conventionally applied with plastic bellow type applicators. Controlled and precise application of powders with this type of delivery system can be challenging, resulting, for example, in the hemostatic agent being blown away from the wound site such that a significant portion of the hemostatic agent can be wasted.

### SUMMARY

One aspect of the disclosure provides a multilayered hemostatic device including a first layer including carboxymethyl cellulose, a second layer including a powdered hemostatic agent provided on the first layer, and a third layer including at least one of gauze, oxidized cellulose, or collagen provided on the second layer, wherein the first layer and the third layer encapsulate the second layer and the hemostatic agent comprises one of fibrinogen, thrombin, fibrin monomers, or the combination of thrombin and gelatin.

Further aspects and advantages will be apparent to those of ordinary skill in the art from a review of the following detailed description. While the methods and compositions are susceptible of embodiments in various forms, the description hereafter includes specific embodiments with the understanding that the disclosure is illustrative, and is not intended to limit the disclosure to the specific embodiments described herein.

### BRIEF DESCRIPTION OF THE DRAWINGS

For further facilitating the understanding of the present invention, two drawing figures are appended hereto.

FIG. 1 shows a rendering of a multilayer device comprising a first soluble/degradable layer, a second powdered hemostatic agent layer, and a third soluble/degradable layer, and also shows after application of the device to a wound such that the first layer is in contact with the wound, the dissolution/degradation of the first layer to permit direct contact of the hemostatic agent with the wound and the subsequent dissolution/degradation of the third layer.

FIG. 2 shows a rendering of a multilayer device comprising a first soluble/degradable layer, a second powdered hemostatic agent layer, and a third non-soluble and non-degradable layer, and also shows after application of the device to a wound such that the first layer is in contact with the wound, the dissolution/degradation of the first layer to permit direct contact of the hemostatic agent with the wound and the subsequent removal of the third layer.

### DETAILED DESCRIPTION

The disclosure provides a multilayered hemostatic device including a first layer including carboxymethyl cellulose, a second layer including a powdered hemostatic agent provided on the first layer, and a third layer including at least one of gauze, oxidized cellulose, or collagen provided on the second layer, wherein the first layer and the third layer encapsulate the second layer and the hemostatic agent comprises one of fibrinogen, thrombin, fibrin monomers, or the combination of thrombin and gelatin. Advantageously, the multilayered hemostatic device of the disclosure allows for the precise, controlled delivery and application of flowable, powdered, hemostatic agents to a wound site in a manner which maximizes the utilization of the powdered hemostatic agent at the surgical site, without waste resulting from imprecise delivery or blowing away of the hemostatic agent from the site of delivery.

As used herein, “powdered hemostatic agent” refers to a flowable, solid particulate material. “Powdered” and “particulate” may be used interchangeably herein.

In embodiments, the powdered hemostatic agent is provided on the first layer in a repeating pattern. In embodiments, the perimeter edges of the first layer and third layer are sealed.

In embodiments, the first layer is soluble under physiological conditions, such that the first layer will dissolve when in contact with a fluid from a wound. Physiological conditions encompass aqueous-based milieu having a temperature in a range of about 20 to about 40° C. and a pH of about 6 to about 8, and an atmospheric pressure of about 0.8 atm to about 1.2 atm and corresponding atmospheric oxygen concentrations. In embodiments, the first layer is degradable under physiological conditions, such that the first layer will degrade when in contact with a fluid from a wound. In embodiments, the third layer is degradable under physiological conditions, such that the third layer will degrade when in contact with a fluid from a wound. In embodiments, the third layer forms a gel under physiological conditions. In embodiments, the third layer is not-soluble and/or not-degradable under physiological conditions, such that the third layer will not dissolve or degrade when in contact with a fluid from a wound and, after application of the device to the wound and dissolution and/or degradation of the first layer, the second layer is disposed between a wound and the third layer when the hemostatic device is applied to the wound.

### First Layer

The device of the disclosure comprises a first layer which is the wound-contacting layer. The first layer may comprise a material that is soluble and/or degradable under physiological conditions, such that the first layer will dissolve and/or degrade when in contact with a fluid from a wound (e.g., blood and/or wound exudate). In embodiments, the first layer is rapidly soluble and/or degradable under physiological conditions and will dissolve and/or degrade after less than about 90 seconds of contact with a fluid from a wound, after less than about 60 seconds of contact with a fluid from a wound, or after less than about 30 seconds of contact with a fluid from a wound.

In embodiments, the first layer comprises carboxymethyl cellulose and may be provided as a knitted, non-woven fabric, as a woven fabric, or as a soluble/degradable film. Other materials for use in addition to or as an alternative to carboxymethyl cellulose include, but are not limited to, polyvinyl alcohols and modified polyvinyl alcohols, polyacrylates, water-soluble acrylate copolymers, polyvinyl pyrrolidone, pullulan, gelatin, hydroxylpropylmethyl cellulose (HPMC), polyethylene oxide, polyethylene glycols, low viscosity grade hydroxypropylcellulose, polysaccharides, water-soluble natural polymers including, but not limited to, guar gum, xanthan gum, locust bean gum, carrageenan, and starch, modified starches including, but not limited to, ethoxylated starch and hydroxypropylated starch, copolymers of the foregoing and combinations of any of the foregoing.

Soluble/degradable films according to the present disclosure may include other optional additive ingredients including, but not limited to, plasticizers, surfactants, film formers, and other functional ingredients, for example in amounts suitable for their intended purpose.

Suitable plasticizers may include glycerol, diglycerol, propylene glycol, ethylene glycol, diethylene glycol, triethylene glycol, tetraethylene glycol, polyethylene glycols up to MW 400, phthalate derivatives, including but not limited to, dimethyl phthalate, diethyl phthalate, and dibutyl phthalate, citrate derivatives, including but not limited to tributyl citrate, triethyl citrate, acetyl citrate, triacetin citrate, and castor oil.

In embodiments, the first layer is soluble under physiological conditions, such that the first layer will dissolve when in contact with a fluid from a wound. In embodiments, the first layer is degradable under physiological conditions, such that the first layer will degrade when in contact with a fluid from a wound.

### Second Layer

The device of the disclosure comprises a second layer comprising a powdered hemostatic agent provided on the first layer. The second, powdered hemostatic agent layer may be a homogenous layer or a heterogeneous layer. In embodiments, the powdered hemostatic agent is provided on the first layer in a repeating pattern, for example, the powdered hemostatic agent can be deposited on the first layer as a pattern of discrete dots, as pattern of discrete columns or rows, or as a repeating pattern of other geometric shapes.

As used herein, “hemostatic agent” refers to a material that exerts a passive or active mode of hemostatic action upon a bleeding wound.

In embodiments, the powdered hemostatic agent is selected from the group consisting of fibrinogen, thrombin, fibrin monomers, gelatin, and mixtures of the foregoing. The powdered hemostatic agent may be a lyophilized hemostatic agent, such as lyophilized thrombin or lyophilized fibrinogen. In embodiments, the powdered hemostatic agent is selected from the group consisting of fibrinogen, thrombin, fibrin monomers, the combination of thrombin and gelatin, and mixtures of the foregoing. In embodiments, the powdered hemostatic agent comprises fibrinogen. In embodiments, the powdered hemostatic agent is fibrinogen. In embodiments, the powdered hemostatic agent comprises fibrin monomers. In embodiments, the powdered hemostatic agent is fibrin monomers. In embodiments, the powdered hemostatic agent comprises thrombin. In embodiments, the powdered hemostatic agent comprises thrombin and gelatin. In embodiments, the powdered hemostatic agent is thrombin. In embodiments, the powdered hemostatic agent is the combination of thrombin and gelatin.

As used herein, “fibrin monomer” includes any form of fibrin, e.g., fibrin I, fibrin II, or des BB fibrin, wherein the fibrin is in monomeric form or oligomeric form that can be solubilized and can be converted to fibrin polymer. Fibrin oligomers are capable of being solubilized under physiological conditions whereas fibrin polymer is not soluble under physiological conditions.

Other powdered/particulate hemostatic agents for use in addition to or as an alternative to fibrinogen, thrombin, fibrin monomer, and gelatin include, but are not limited to, oxidized cellulose, chitosan, alginate, polysaccharides, ethyl cellulose beads and beads of other oxidized celluloses, maltodextrin, clotting factor concentrates, recombinant Factor Vila, alphanate FVIII concentrate, bioclate FVm concentrate, monoclate-P FVHI concentrate, haemate P FVIII, von Willebrand factor concentrate, helixate FVII concentrate, kogenate FVII concentrate, recombinate FVIII concentrate, mononine FIX concentrate, fibrogammin P FXIII concentrate, and combinations of the foregoing.

### Third Layer

The device of the disclosure comprises a third layer provided on the second layer, wherein the first layer and the third layer encapsulate the second layer. In embodiments, the third layer may be soluble and/or degradable under physiological conditions, such that the third layer will dissolve and/or degrade when in contact with a fluid from a wound. In embodiments, the third layer may be non-soluble and non-degradable under physiological conditions, such that the third layer will not dissolve or degrade when in contact with a fluid from a wound, even when the contact between the wound and the third layer is for an extended period of time, for example, a period of time greater than 5 minutes, 15 minutes, or even one hour. In embodiments, the third layer may form a gel under physiological conditions.

In embodiments, the third layer comprises a material selected from the group consisting of gauze, oxidized cellulose, collagen, and mixtures of the foregoing. In embodiments, the third layer comprises gauze. In embodiments, the third layer is gauze. In embodiments, the third layer comprises oxidized cellulose. In embodiments, the third layer is oxidized cellulose. In embodiments, the third layer comprises collagen. In embodiments, the third layer is collagen. Optionally, the oxidized cellulose is oxidized regenerated cellulose.

Suitable other materials for use in addition to or as an alternative to gauze, oxidized cellulose, and collagen include, but are not limited to, silicone, keratin, gelatin, chitin, chitosan, polyvinyl pyrrolidone, alginate, cross-linked polyvinyl alcohol, and combinations of the foregoing.

The third layer may also optionally include a pH modifier. Without intending to be bound by theory, it is believed that when the second layer comprises thrombin and the third layer comprises oxidized cellulose or oxidized regenerated cellulose, the acidic pH of the third layer may result in precipitation of fibrinogen from the wound before it has a chance to react with the thrombin to form a clot. Accordingly, without intending to be bound by theory, a pH modifier may be included in the third layer to provide the third layer with a neutral pH to avoid the precipitation of fibrinogen. Suitable pH modifiers include, but are not limited to, soda ash, sodium silicate, lime, sodium phosphate, and buffer compositions, for example, acetate buffers, citrate buffers, phosphate-buffered saline. pH modifiers can be provided in particulate form or can be coated on the third layer, for example, as a solution.

The device of the disclosure may have any suitable configuration, including but not limited to, triangular, quadrilateral, such as square or rectangular, circular, oblong, pentagon, hexagon, and octagon, for example. In embodiments, at least the first layer and the third layer have a similar configuration such that the first layer and third layer have substantially parallel perimeter edges to facilitate encapsulation and sealing of the second layer between the first and third layers.

In embodiments, the perimeter edges of the first layer and the third layer may be sealed. The perimeter edges of the first layer and third layer may be sealed according to any suitable process known in the art, including but not limited to, solvent sealing and heat sealing.

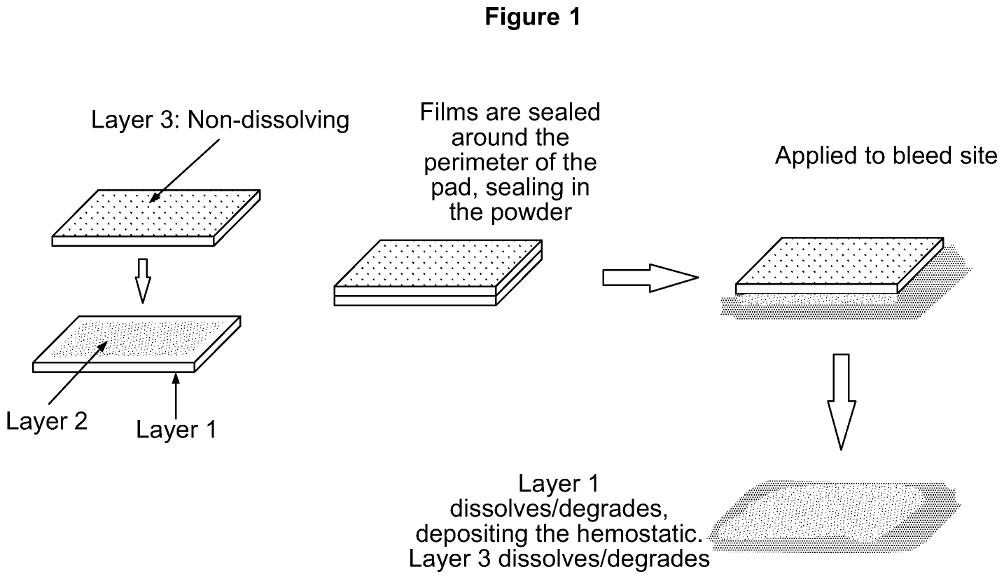

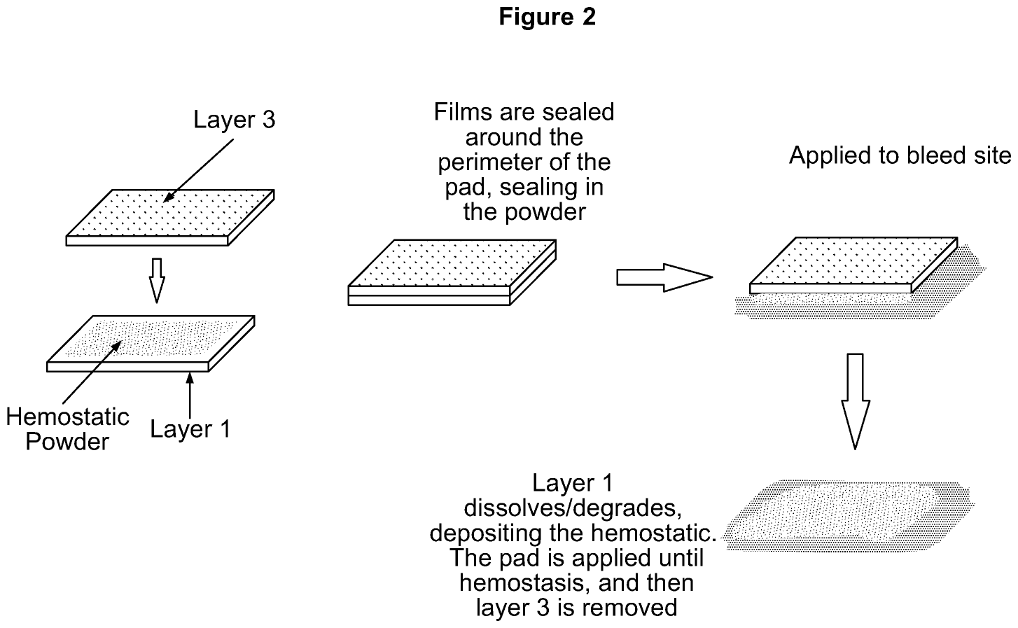

As shown in FIG. 1 and FIG. 2, the device of the disclosure may be disposed on a wound to provide a precise delivery and application of powdered hemostatic agents. Advantageously, after the device is applied such that the first layer is in contact with the wound, the first wound-facing layer readily dissolves and/or degrades such that the hemostatic agent of the second layer is subsequently disposed between, and in contact with, the wound and the third layer. The third layer may be selected such that it will not dissolve or degrade until hemostasis of the wound has occurred or such that it will not dissolve or degrade at all and will remain in place until hemostasis of the wound has occurred and the third layer is removed.

Accordingly, in embodiments, the rate of dissolution and/or degradation of the first layer under physiological conditions is greater than the rate of degradation and/or dissolution of the third layer under physiological conditions. Further, in embodiments, the third layer is not-soluble and/or not-degradable under physiological conditions and the second layer is disposed between a wound and the third layer after the hemostatic device is applied to the wound.

Alternatively, the third layer may be selected to readily dissolve and/or degrade after application to a wound and subsequent dissolution/degradation of the first layer and delivery of the hemostatic agent of the second layer to the wound, such that the third layer comes into contact with the wound.

In embodiments, when the hemostatic agent comprises fibrinogen, the third layer comprises collagen.

The combinations provided in Table 1, below, are each particularly contemplated embodiments. In each of the combinations provided in Table 1, it is specifically contemplated that the first layer, second layer, and third layer may comprise the listed materials or may consist of the listed materials.

TABLE 1

| | First Layer | Second Layer | Third Layer |
|---|---|---|---|
| 1 | Carboxymethyl | Thrombin and gelatin | Oxidized cellulose |
| | cellulose | | |
| 2 | Carboxymethyl | Thrombin and gelatin | Gauze |
| | cellulose | | |
| 3 | Carboxymethyl | Thrombin and gelatin | Collagen |
| | cellulose | | |
| 4 | Carboxymethyl | Fibrinogen | Oxidized regenerated |
| | cellulose | | cellulose |
| 5 | Carboxymethyl | Thrombin | Oxidized regenerated |
| | cellulose | | cellulose |
| 6 | Carboxymethyl | Fibrin monomers | Oxidized regenerated |
| | cellulose | | cellulose |

The foregoing description is given for clearness of understanding only, and no unnecessary limitations should be understood therefrom, as modifications within the scope of the invention may be apparent to those having ordinary skill in the art.

All patents, publications, and references cited herein are hereby fully incorporated by reference. In case of a conflict between the present disclosure and incorporated patents, publications, and references, the present disclosure should control.

What is claimed:

1. A multilayered hemostatic device comprising:
a first layer comprising carboxymethyl cellulose;
a second layer comprising a powdered hemostatic agent provided on the first layer; and
a third layer comprising at least one of gauze, oxidized cellulose, or collagen provided on the second layer, wherein the first layer and the third layer encapsulate the second layer,
wherein the hemostatic agent comprises one of fibrinogen, thrombin, fibrin monomers, or the combination of thrombin and gelatin.

2. The multilayered hemostatic device of claim 1, wherein the powdered hemostatic agent is a solid particulate.

3. The multilayered hemostatic device of claim 1 or claim 2, wherein the powdered hemostatic agent is provided on the first layer in a repeating pattern.

4. The multilayered hemostatic device of any one of the preceding claims, wherein the first layer and the third layer have perimeter edges and the first layer and third layer are sealed along the perimeter edges.

5. The multilayered hemostatic device of claim 4, wherein the perimeter edges of the first layer and the third layer are heat sealed.

6. The multilayered hemostatic device of claim 4, wherein the perimeter edges of the first layer and the third layer are solvent sealed.

7. The multilayered hemostatic device of any one of the preceding claims, wherein the first layer is soluble under physiological conditions, such that the first layer will dissolve when in contact with a fluid from a wound.

8. The multilayered hemostatic device of any one of the preceding claims, wherein the rate of dissolution of the first layer under physiological conditions is greater than the rate of degradation of the third layer under physiological conditions.

9. The multilayered hemostatic device of any one of the preceding claims, wherein the third layer forms a gel under physiological conditions.

10. The multilayered hemostatic device of any one of the preceding claims, wherein the third layer is not-soluble and/or not-degradable under physiological conditions and the second layer is disposed between a wound and the third layer when the hemostatic device is applied to the wound.

11. The multilayered hemostatic device of any one of the preceding claims, wherein the oxidized cellulose is oxidized regenerated cellulose.

12. The multilayered hemostatic device of any one of the preceding claims, wherein the third layer comprises oxidized cellulose, and the hemostatic agent comprises the combination of thrombin and gelatin.

13. The multilayered hemostatic device of any one of claims 1 to 10, wherein the third layer comprises gauze, and the hemostatic agent comprises the combination of thrombin and gelatin.

14. The multilayered hemostatic device of any one of claims 1 to 10, wherein the third layer comprises collagen, and the hemostatic agent comprises the combination of thrombin and gelatin.

15. The multilayered hemostatic device of any one of claims 1 to 11, wherein the third layer comprises oxidized regenerated cellulose, and the hemostatic agent comprises fibrinogen.

16. The multilayered hemostatic device of any one of claims 1 to 11, wherein the third layer comprises oxidized regenerated cellulose, and the hemostatic agent comprises thrombin.

17. The multilayered hemostatic device of any one of claims 1 to 11, wherein the third layer comprises oxidized regenerated cellulose, and the hemostatic agent comprises fibrin monomers.

18. The multilayered hemostatic device of any one of claim 12, 13, or 14, wherein the hemostatic agent is the combination of thrombin and gelatin.

19. The multilayered hemostatic device of claim 15, wherein the hemostatic agent is fibrinogen.

20. The multilayered hemostatic device of claim 16, wherein the hemostatic agent is thrombin.

21. The multilayered hemostatic device of claim 17, wherein the hemostatic agent is fibrin monomers.

22. The multilayered hemostatic device of any one of claims 1 to 10, wherein the first layer comprises at least one of carboxymethyl cellulose, polyvinyl alcohols and modified polyvinyl alcohols, polyacrylates, water-soluble acrylate copolymers, polyvinyl pyrrolidone, pullulan, gelatin, hydroxylpropylmethyl cellulose (HPMC), polyethylene oxide, polyethylene glycols, low viscosity grade hydroxypropylcellulose, polysaccharides, water-soluble natural polymers including, but not limited to, guar gum, xanthan gum, locust bean gum, carrageenan, and starch, modified starches including, but not limited to, ethoxylated starch and hydroxypropylated starch, copolymers of the foregoing and combinations of any of the foregoing;
the second layer comprises at least one of fibrinogen, thrombin, fibrin monomer, gelatin, oxidized cellulose, chitosan, alginate, polysaccharides, ethyl cellulose beads and beads of other oxidized celluloses, maltodextrin, clotting factor concentrates, recombinant Factor Vila, alphanate FVIII concentrate, bioclate FVm concentrate, monoclate-P FVHI concentrate, haemate P FVIII, von Willebrand factor concentrate, helixate FVII concentrate, kogenate FVII concentrate, recombinate FVIII concentrate, mononine FIX concentrate, fibrogammin P FXIII concentrate, and combinations of the foregoing; and
the third layer comprises at least one of gauze, oxidized cellulose, collagen, silicone, keratin, gelatin, chitin, chitosan, polyvinyl pyrrolidone, alginate, cross-linked polyvinyl alcohol, and combinations of the foregoing.
